(19) United States
(12) Patent Application Publication
(10) Pub. No.: US 2015/0359729 A1
(43) Pub. Date: Dec. 17, 2015

(54) Lip Treatment Composition

(71) Applicant: Ronald Anderson, Sylmar, CA (US)

(72) Inventor: Ronald Anderson, Sylmar, CA (US)

(21) Appl. No.: 14/738,002

(22) Filed: Jun. 12, 2015

Related U.S. Application Data
(60) Provisional application No. 62/012,730, filed on Jun. 16, 2014.

(51) Int. Cl. A61K 8/97; A61Q 19/00; A61K 8/92

(52) U.S. Cl. CPC A61K 8/97; A61K 8/922; A61K 8/925; A61Q 19/001

(57) ABSTRACT

A lip treatment composition for creating a perceptible tingling sensation when applied to mucosa membrane of a user. The composition has one or more base ingredients and at least one active ingredient, such as Zanthoxylum essential oil, that induces a perceptible tingling sensation reminiscent of electrical buzzing when applied to the mucosa membrane of the user. The Zanthoxylum essential oil could comprises between approximately 2.0000% and 4.0000% by weight of the composition and could comprise approximately 3.3000% by weight of the composition. The lip treatment composition can be formed under a process including heating the one or more base ingredients, adding the at least one active ingredient, and mixing the one or more base ingredients and the at least one active ingredient.

### FIELD OF THE INVENTION

The present invention relates generally to lip treatment products. Stated more particularly, disclosed herein is a lip treatment composition that, when applied directly to the lips, causes a perceptible sensation, such as a tingling sensation reminiscent of electrical buzzing and pulsing, across the surface of the lips.

### BACKGROUND OF THE INVENTION

Numerous pomades for lips have been disclosed by the prior art. Indeed, new disclosures for lip balms, lip gloss, and lipsticks are continually added to the multi-billion dollar cosmetics industry. By way of example and not limitation, lip balms and other lip treatment products demonstrating new colors, textures, and overall compositions are regularly reformulated for new trends and new generations of users.

For the most part, prior art lip treatment products have been provided for aesthetic purposes and, additionally or alternatively, to soothe and smooth the user's lips. Functionally speaking, therefore, there has been little real separation between the many lip balms and other lip treatments taught by even the voluminous prior art. Few, if any, disclosed lip products provide or even seek to provide exceptional, perceivable sensations and feelings to the lips of the user. Indeed, it can be difficult even for experienced users to distinguish between the many lip treatments of the prior art.

The present inventor has thus recognized that would be advantageous to provide a lip treatment that is of unique composition and performance that provides a perceptible sensation to the lips of the user such that the user realizes a phenomenal hp treatment experience.

### SUMMARY OF THE INVENTION

With a knowledge of the state of the art and the limitations and deficiencies thereof as summarized above, the present inventor set forth with the basic object of providing a lip treatment composition that presents a viable solution to the disadvantages of the prior art while providing a plurality of further improvements thereover.

A more particular object of embodiments of the invention is to provide a lip treatment of unique composition and performance that provides a perceptible tingling sensation to the mucosa membrane, such as the lips, of the user as when applied in a coating.

A more particular object of embodiments of the invention is to provide a lip treatment composition formed entirely from natural ingredients.

A further object of embodiments of the invention is to provide a lip treatment composition with one or more active ingredients that, when applied directly to the lips or other mucosa membrane, creates a tingling sensation, potentially a perception of neurological confusion, that induces a tingling sensation across the surface of the lips.

A more particular object of embodiments of the invention is to provide a lip treatment composition that creates a tingling sensation across the surface of the lips that may vary for different individuals but that typically begins within a short time period after application of the composition and persists for an appreciable time period thereafter.

A further object of embodiments of the invention is to provide a lip treatment composition that creates a sensation reminiscent of electrical buzzing and pulsing.

Still another object of embodiments of the invention is to provide a lip treatment composition that creates a sensation reminiscent of electrical buzzing and pulsing that intensifies in proportion to the amount of composition applied directly to the lips.

Still another object of embodiments of the invention is to provide a lip treatment composition capable of sharing and partially transferring the induced tingling sensation to other persons by physical contact, such as kissing.

These and further objects and advantages of embodiments of the invention will become obvious not only to one who reviews the present specification but also to one who has an opportunity to make use of an embodiment of the lip treatment composition disclosed herein. It will be appreciated, however, that, although the accomplishment of each of the foregoing objects in a single embodiment of the invention may be possible and indeed preferred, not all embodiments will seek or need to accomplish each and every potential object and advantage. Nonetheless, all such embodiments should be considered within the scope of the invention.

In one embodiment, a lip treatment composition capable of producing a perceptible tingling sensation when applied to mucosa membrane of a user comprises one or more base ingredients in combination with at least one active ingredient selected to induce a perceptible tingling sensation when the lip treatment composition is applied to the mucosa membrane of the user. The tingling or buzzing sensation may vary in specific timing and effect for different individuals. The sensation can begin within a short time period after application of the lip treatment and can persist for an appreciable time period thereafter. In certain practices of the invention, the perceived sensation can be reminiscent of electrical buzzing and pulsing, and that sensation can intensify in proportion to the amount of composition applied. The disclosed lip treatment composition could be shared, such as by physical contact between the lips of a user of the composition and the lips of a second person.

The at least one active ingredient could comprise Zanthoxylum, potentially in the form of Zanthoxylum essential oil. Where the at least one active ingredient comprises Zanthoxylum essential oil, it could comprise between approximately 2.0000% and 4.0000% by weight of the composition. Even more particularly, Zanthoxylum essential oil could comprise approximately 3.3000% by weight of the composition. In certain practices of the invention, the at least one active ingredient could further comprise ginger essential oil and cinnamon bark oil. As where the at least one active ingredient comprises Zanthoxylum essential oil, the perceptible tingling sensation can be in the form of a perception of an electrical buzzing and pulsing sensation on mucosa membrane to which the lip treatment composition is applied.

While the lip treatment composition could comprise a lipstick, a lip gloss, or any other lip treatment, embodiments of the lip treatment composition are contemplated as comprising a lip balm. In such practices of the invention, the one or more base ingredients could include white beeswax. In any event, the lip treatment composition could further comprise at least one flavoring ingredient and, potentially, at least one coloring ingredient.

The lip treatment composition could be formed under a process comprising the steps of heating the one or more base ingredients, adding the at least one active ingredient to the one or more base ingredients after the step of heating the one or more base ingredients, and mixing the one or more base ingredients and the at least one active ingredient. The step of mixing the one or more base ingredients and the at least one active ingredient could, for instance, be performed in a rotational mixer. The method could further comprise heating the one or more base ingredients and the at least one active ingredient after the step of mixing prior to separation and packaging of the composition so formed.

One will appreciate that the foregoing discussion broadly outlines the more important features of the invention merely to enable a better understanding of the detailed description that follows and to instill a better appreciation of the inventor's contribution to the art. Before an embodiment of the invention is explained in detail, it must be made clear that the following details and descriptions of inventive concepts are mere examples of the many possible manifestations of the invention.

### DETAILED DESCRIPTION OF PREFERRED EMBODIMENTS

The lip treatment composition disclosed herein is subject to varied embodiments, each within the scope of the invention. However, to ensure that one skilled in the art will be able to understand and, in appropriate cases, practice the present invention, certain preferred embodiments of the broader invention revealed herein are described below.

With the foregoing in mind, manifestations of the lip treatment composition pursuant to the present invention could, by way of example and not limitation, be founded on base ingredients, including flavors. The base ingredients could be standard and commonly used for the type of lip treatment composition at hand. It will again be understood that the lip treatment composition could comprise substantially any type of lip treatment composition, including but not limited to lip balm, a lipstick, lip gloss, or some other lip coating.

One presently contemplated composition embodying the invention takes the form of a lip balm. There, the lip treatment composition could be formed with base ingredients, such as white beeswax and sweet almond oil. For example, the white beeswax could be provided at approximately 20.0000% by weight, and the sweet almond oil could be provided at approximately 66.5000% by weight.

With base ingredients, the composition can further include active ingredients. As taught herein, the active ingredients could include Zanthoxylum, such as a Zanthoxylum essential oil, for producing the desired tingling physical sensation reminiscent of electrical buzzing. The Zanthoxylum essential oil could, in preferred embodiments, be included at between approximately 2.0000% and 4.0000% by weight. For the desired buzzing effect of the present invention, the Zanthoxylum essential oil may most preferably be provided at approximately 3.3000% by weight. The active ingredients could further include one or more of ginger essential oil, cinnamon bark oil, sweet orange extract, vitamin E, and rosemary oil extract. For instance, additional active ingredients could be 0.5000% ginger essential oil, 0.4000% cinnamon bark oil 18J, 0.5000% sweet orange extract, 0.2000% T50 natural vitamin E, and 0.1000% rosemary oil extract, all by weight.

The composition could further include flavoring and coloring ingredients. By way of example and not limitation, a lip treatment composition as taught herein could be a strawberry composition. To achieve a composition that might be characterized as a strawberry composition, such an embodiment could further include approximately strawberry flavoring and red coloring. For example, the flavoring ingredient could be 8.000% by weight strawberry 416041SU R517913 (natural), and the coloring could be approximately 0.5000% by weight red oil-based candy color. Of course, strawberry flavoring and corresponding coloring are mere examples. Other flavors and colorings will be readily possible using the foregoing teachings and should be considered as part of this disclosure.

So disclosed, the lip treatment composition according to the invention can be completely natural in ingredients. The composition can include essential oils. Moreover, it is functional for its intended purposes without a need for menthol or mint. It will be understood, though, that one or more such ingredients could be included within the scope of the invention and should not be considered as excluded except as might be expressly required by the claims.

It will be understood that the lip treatment composition could include additional active or inactive ingredients. Accordingly, although the foregoing components and proportions of components may indeed be preferable and advantageous in achieving one or more objects of the invention, the lip treatment composition shall not be interpreted to require all of the foregoing components, to be limited to the specified components, or to be limited even to the approximate proportions of components except as the claims might expressly specify. By way of example, fewer, additional, or different ingredients or ingredients in different proportions could be provided to create different flavors or effects or otherwise within the scope of the invention.

Finished lip treatment compositions could be created according to a particularized process. The process could begin with heating the base ingredient(s), such as the white beeswax, which again could comprise roughly 20.0000% by volume of the overall composition, to melting. The ingredients of the lip treatment composition can then be combined, potentially while continuously or periodically mixing the components. For instance, the components could be mixed in a rotational mixer. The mixer could, for example, spin at a rate of approximately 300 revolutions per minute (RPM). With the components rotating at a given rotational rate, such as approximately 300 RPM, the composition can be heated to an elevated temperature, such as approximately 60° C. (140° F.). So combined and heated, the composition can be dispensed to suitable containers for distribution and use or, potentially, for further separating and packaging.

The lip treatment composition so disclosed can be applied by a user to the mucosa membrane, such as the lips, of a human. The lip treatment composition seeks to provide unique performance with a perceptible tingling sensation to the lips of the user, potentially while being formed entirely from natural ingredients. The lip treatment composition has one or more active ingredients that, when applied directly to the lips or other mucosa membrane, seeks to create a tingling sensation, potentially a perception of neurological confusion, that induces a sensation reminiscent of electrical buzzing across the surface of the lips.

Although the tingling or buzzing sensation may vary in specific timing and effect for different individuals, the sensation typically begins within a short time period after application of the lip treatment and persists for an appreciable time period thereafter. By way of example and not limitation, the sensation could begin within approximately thirty to ninety seconds of application and can last for approximately ten to fifteen minutes. The perceived sensation can be characterized as being reminiscent of electrical buzzing and pulsing, and that sensation can intensify in proportion to the amount of composition applied directly to the lips. Moreover, the lip treatment composition disclosed herein is capable of permitting sharing and partially transferring the induced sensation to other persons by physical contact between the lips or another body surface of a first person to which the composition has been applied and the lips or another body surface of a second person. For example, the composition and its effect could be transferred by kissing.

With certain details and embodiments of the present invention for a lip treatment composition disclosed, it will be appreciated by one skilled in the art that numerous changes and additions could be made thereto without deviating from the spirit or scope of the invention. This is particularly true when one bears in mind that the presently preferred embodiments merely exemplify the broader invention revealed herein. Accordingly, it will be clear that those with major features of the invention in mind could craft embodiments that incorporate those major features while not incorporating all of the features included in the preferred embodiments.

Therefore, the following claims shall define the scope of protection to be afforded to the patent holder. Those claims shall be deemed to include equivalent constructions insofar as they do not depart from the spirit and scope of the invention. It must be further noted that a plurality of the following claims may express certain elements as means for performing a specific function, at times without the recital of structure or material. As the law demands, any such claims shall be construed to cover not only the corresponding structure and material expressly described in this specification but also all equivalents thereof.

I claim as deserving the protection of Letters Patent:

1. A lip treatment composition for creating a perceptible tingling sensation when applied to mucosa membrane of a user, the lip treatment composition comprising:
one or more base ingredients; and
at least one active ingredient selected to induce a perceptible tingling sensation when the lip treatment composition is applied to the mucosa membrane of the user.

2. The lip treatment composition of claim 1 wherein the at least one active ingredient comprises Zanthoxylum.

3. The lip treatment composition of claim 2 wherein the at least one active ingredient further comprises ginger essential oil and cinnamon bark oil.

4. The lip treatment composition of claim 2 wherein the at least one active ingredient comprises Zanthoxylum essential oil.

5. The lip treatment composition of claim 4 wherein the Zanthoxylum essential oil comprises between approximately 2.0000% and 4.0000% by weight of the composition.

6. The lip treatment composition of claim 5 wherein the Zanthoxylum essential oil comprises approximately 3.3000% by weight of the composition.

7. The lip treatment composition of claim 2 wherein the perceptible tingling sensation comprises a perception of an electrical buzzing and pulsing sensation on mucosa membrane to which the lip treatment composition is applied.

8. The lip treatment composition of claim 1 wherein the lip treatment composition comprises a lip balm and wherein the one or more base ingredients include white beeswax.

9. The lip treatment composition of claim 1 further comprising at least one flavoring ingredient and at least one coloring ingredient.

10. The lip treatment composition of claim 1 wherein the lip treatment composition is formed under a process comprising the following steps:
heating the one or more base ingredients;
adding the at least one active ingredient to the one or more base ingredients after the step of heating the one or more base ingredients; and
mixing the one or more base ingredients and the at least one active ingredient.

11. The lip treatment composition of claim 10 wherein the step of mixing the one or more base ingredients and the at least one active ingredient is performed in a rotational mixer.

12. The lip treatment composition of claim 11 further comprising the step of heating the one or more base ingredients and the at least one active ingredient after the step of mixing.
